(19) United States
(12) Patent Application Publication
(10) Pub. No.: US 2026/0297344 A1
(43) Pub. Date: Oct. 1, 2026

(54) BIOINK COMPOSITIONS FOR VISIBLE LIGHT CURING, METHOD OF MANUFACTURING THE SAME, AND METHOD OF PRINTING THE SAME

(71) Applicant: POSTECH Research and Business Development Foundation, Pohang-si, Gyeongsangbuk-do (KR)

(72) Inventors: Jinah JANG, Pohang-si (KR); Hyeon Ji KIM, Pohang-si (KR); Dong Woo CHO, Pohang-si (KR); Byeongmin KANG, Pohang-si (KR); Daekeun KIM, Pohang-si (KR)

(73) Assignee: POSTECH Research and Business Development Foundation, Pohang-si, Gyeongsangbuk-do (KR)

(21) Appl. No.: 18/998,223

(22) PCT Filed: Sep. 6, 2023
(86) PCT No.: PCT/KR2023/013308

§ 371 Date: Jan. 24, 2025

(30) Foreign Application Priority Data
Sep. 16, 2022 (KR) 10-2022-0116901
Aug. 28, 2023 (KR) 10-2023-0112689

(51) Int. Cl. C09D 11/03; A61L 27/36; C09D 11/04

(52) U.S. Cl. CPC C09D 11/03; A61L 27/3633; A61L 27/3683; C09D 11/04

(57) ABSTRACT

The present invention relates to bioink compositions for visible light curing, method of manufacturing the same, and method of printing the same. More particularly, the bioink composition comprises an extracellular matrix (dECM) obtained by treating decellularized tissue isolated from a living body and ruthenium/sodium persulfate (Ru/SPS) as a photoinitiator, which is photocrosslinked by visible light irradiation. This composition exhibits excellent mechanical properties due to physical crosslinking through intermolecular interactions, along with superior cell compatibility and high shape retention.

### TECHNICAL FIELDS

The present invention relates to bioink compositions, and more particularly, to bioink compositions for visible light curing for 3D (three-dimensional) printing. These compositions exhibit excellent mechanical properties, high shape retention, and superior cell compatibility through physical crosslinking via intermolecular interactions. The invention also relates to methods for manufacturing such bioink compositions, methods for printing such bioink compositions, and methods for manufacturing tissue mimics using such bioink compositions.

### BACKGROUND ARTS

The field of tissue engineering and regenerative medicine offers great opportunities to create transplantable functional tissue structures to overcome the shortage of organs. This field is also applicable to the development of in vitro tissue models for studying disease mechanisms, drug screening, new drug development, and toxicity testing. Previously, most methods have involved transplanting functional biomaterials that were simply seeded with cells or fortified with bioactive elements, or injecting cells into these biomaterial-based implants. However, there are great difficulties in producing large and complex 3D structures such as corneas or heart ventricles, which limits the supply of alternative organs.

3D bioprinting offers precise control, enabling the arrangement of cells and materials into desired structures at specified location. This technology provides excellent process flexibility, facilitating the fabrication of tissue-specific cell arrangements and various structural features, especially in the field of tissue engineering. With advances in the bioink manufacturing technology, there is also the possibility of enhancing the functionality of the engineered tissue constructs produced in this way. However, existing bioinks face limitations in achieving multi-scale control necessary for producing clinically applicable human-scale tissue mimics. These limitations arise due to the trade-off between shape retention and biocompatibility. Furthermore, it is essential to reproduce the tissue-specific microenvironment, which include intrinsic biochemical signals essential for supporting various cellular behaviors. Consequently, there is still a high demand for the development of multifunctional bioinks that enable straightforward process control while reproducing the biophysical/biochemical microenvironments required for fabricating 3D tissue models.

Meanwhile, decellularized extracellular matrix (dECM) is considered a biomaterial for mimicking and reproducing the complex microenvironment within tissue-mimicking scaffolds, as it contains biomolecular components and composition similar to those of actual tissues and organs. Unlike existing natural bioinks (collagen, silk fibroin, gelatin, etc.), which are composed of a single component, dECM can provide tissue-specific factors and a microscale structure that contribute to cell specification, differentiation, and tissue morphogenesis.

In particular, dECM-based bioinks offer the advantage of being used as a thermo-crosslinkable hydrogel through physical crosslinking via molecular interactions of the abundant protein collagen within. In addition, the microscale physical structure in the biochemical environment of dECM-based bioink facilitates the regulation of various cellular behaviors and supports the extracellular matrix (ECM) for tissue regeneration.

Despite these developments, dECM-based bioinks remain insufficiently printable and mechanically stable, limiting their practical application in the production of 3D tissue models. Therefore, various strategies have been explored to increase shape retention by using a support layer or sacrificial layer for the production of clinically applicable large-volume tissue models. Although these strategies can produce structures with large volumes and complex shapes, they require post-printing material processing, including the removal of support or sacrificial layers, which poses a risk of collapse for the produced tissue constructs.

Other methods, such as adding external crosslinkers to dECM-based bioink, or incorporating photocrosslinking processes through chemical modification of dECM-based bioink, have also been attempted. However, these methods are limited by the inevitable reduction in the inherent bioactivity of dECM.

Recently, the dECM-based photocrosslinking methods for bio-ink, which induce the formation of crosslinks between substrate molecules or proteins present in the dECM, has been adopted. The photoinitiator riboflavin, clinically approved and used for treating corneal damage, has been applied for such processes. However, the light source used to trigger the crosslinking reaction is UVA (365 nm), which has been reported to be cytotoxic and mutagenic, potentially causing damage to all substrates. In addition, the photocrosslinking method using riboflavin and UVA suffers from slow crosslinking speed and relatively low efficiency, both of which are critical for producing large-volume tissue mimics.

### RELATED TECHNICAL DOCUMENTS

### Patent Documents

Korean registered patent No. 10-2145594 (Published Date: Aug. 18, 2020)

### SUMMARY OF THE INVENTION

### Technical Subject

The present invention is provided to solve the aforementioned problems and aims to provide a bioink composition with excellent mechanical properties and shape retention through physical crosslinking via intermolecular interactions, as well as excellent cell compatibility.

### Technical Solution

One exemplary embodiment of the present invention may comprise a method for manufacturing bioink compositions, comprising the following steps of: preparing an extracellular matrix (ECM) by treating tissue separated from a living body with a decellularization process; manufacturing decellularized Extracellular matrix Ruthenium Sodium Persulfate (dERS) by mixing ruthenium/Sodium persulfate (Ru/SPS) as a photoinitiator with the prepared decellularized extracellular matrix (dECM); and irradiating visible light onto the manufactured decellularized Extracellular matrix Ruthenium Sodium persulfate (dERS) to photocrosslink the same.

In one general aspect of the present invention, the present invention may be that di-tyrosine bonding is generated between tyrosine residues in the extracellular matrix (dECM) through the above-mentioned visible light irradiation.

Preferably, but not necessarily, it is possible to mix ruthenium/sodium persulfate (Ru/SPS) as the photoinitiator with the decellularized extracellular a matrix (dECM) at concentration ranging from 0.5/5 mM to 10/100 mM, and it is desirable to mix it at a concentration ranging from 1/10 mM to 2/20 mM.

Preferably, but not necessarily, the visible light may have a wavelength in the range of 400 nm to 450 nm, and in some case, it may have a wavelength in the range of 400 nm to 425 nm.

Preferably, but not necessarily, the visible light irradiation may be performed for a period ranging from 5 seconds to 10 minutes.

Preferably, but not necessarily, the irradiation of the visible light may be performed for a time ranging from 1 minute to 5 minutes.

Preferably, but not necessarily, the irradiation of the visible light may be performed for a period of 3 minutes.

Furthermore, the present invention may include an additional step of thermally crosslinking the above-mentioned photocrosslinked extracellular matrix (dERS).

In another general aspect of the present invention, there may be provided a printing method of a bioink composition, comprising the following steps of: preparing an extracellular matrix (ECM) by treating tissue separated from a living body with a decellularization process; preparing an extracellular matrix for photo crosslinking (dERS) by mixing the prepared decellularized extracellular matrix (dECM) with ruthenium/sodium persulfate (Ru/SPS) as a photoinitiator; and printing the above-mentioned photocrosslinkable extracellular matrix (dERS) in an environment where visible light is irradiated.

Preferably, but not necessarily, di-tyrosine bonding can be generated between tyrosine residues in the decellularized extracellular matrix (dECM) through the irradiation of visible light.

In still another general aspect of the present invention, there may be a bioink composition, comprising an extracellular matrix (dECM) obtained by treating decellularized tissue isolated from living a body with a photoinitiator, ruthenium/sodium persulfate (Ru/SPS), and capable of being photocrosslinked by visible light irradiation.

Preferably, but not necessarily, di-tyrosine bonding may be generated between tyrosine residues in the decellularized extracellular matrix (dECM) by the above-mentioned visible light irradiation.

Preferably, but not necessarily, the ruthenium/sodium persulfate (Ru/SPS) may be included in the decellularized extracellular matrix (dECM) at a concentration ranging from 1/10 mM to 10/100 mM.

In a further aspect of the present invention, there may be provided a method for producing a tissue mimic, which includes the step of printing the above-mentioned bioink composition in an environment where visible light is irradiated.

Preferably, but not necessarily, the visible light may have a wavelength in the range of 400 nm to 450 nm.

The specific details of other embodiments are included in the detailed description and drawings.

### Advantageous Effects

The present invention uses ruthenium/sodium persulfate (Ru/SPS) as a photoinitiator in decellularized extracellular matrix (dECM) and crosslinks it with visible light, thereby advantageously producing di-tyrosine bonding between tyrosine residues in the decellularized extracellular matrix (dECM).

This bioink composition has excellent mechanical properties due to physical crosslinking through intermolecular interactions. It also demonstrates excellent cell compatibility as well as high shape retention.

### BRIEF DESCRIPTION OF DRAWINGS

FIG. 1 shows the gelation characteristics results from photocrosslinking using Ru/SPS ( 2/20 mM) as photoinitiator, compared with LAP, Irgacure 2959, and riboflavin, according to an exemplary embodiment of the present invention.

FIG. 2 shows the LC-MS/MS analysis results of tyrosine groups with or without photo crosslinking according to an exemplary embodiment of the present invention (**p<0.01).

FIG. 3 is a self-luminescence photograph of dECM and dERS, demonstrating di-tyrosine crosslinking initiated by crosslinkRu/SPS according to an exemplary embodiment of the present invention.

FIG. 4 shows the fluorescence intensity results of dECM and dERS bio-ink, indicating the di-tyrosine crosslinking effected initiated by crosslinkRu/SPS according to an exemplary embodiment of the present invention (***p<0.0001).

FIG. 5 is the sol fraction result, indicating the di-tyrosine crosslinking effected initiated by crosslinkRu/SPS according to an exemplary embodiment of the present invention (*p<0.1).

FIG. 6 is the result of swelling ratio, demonstrating the di-tyrosine crosslinking effected initiated by crosslinkRu/SPS according to an exemplary embodiment of the present invention (*p<0.1, **p<0.01).

FIG. 7 shows the crosslinking time results for the photo-activated and thermally crosslinked dECM hydrogel, as an outcome of the di-tyrosine crosslinking effect initiated by Ru/SPS according to an exemplary embodiment of the present invention.

FIG. 8 shows the results of increased storage and loss moduli after exposure to light for three minutes (light source: visible light (30 mW cm⁻² post-printing conditions)), illustrating the rheological properties of bioink before and after printing using 2% hdECM and 2% Co-dECM, according to an exemplary embodiment of the present invention.

FIG. 9 shows the rheological properties of bioink before and after printing using 2% hdECM and 2% Co-dECM, demonstrating the mechanical property improvement of the compression modulus due to di-tyrosine synthesis following photoactivation and thermal crosslinking process, according to an exemplary embodiment of the present invention (*p<0.1, **p<0.01).

FIGS. 10 to 12 show, according to an exemplary embodiment of the present invention, the results of 3D printing with a high-aspect-ratio structure at the centimeter level using a light-activated crosslinking system and dERS, wherein,

FIG. 10 is a comparative photograph of products manufactured using dECM and dERS with or without a light-activated crosslinking system, as an example of the present invention,

FIG. 11 shows the height of the product in FIG. 10 (**p<0.01), and

FIG. 12 shows the width of the cylindrical structure in the product in FIG. 10 (****p<0.0001) (scale bar: 10 mm, VIS: visible light).

FIGS. 13 and 14 depict the structure of a corneal stroma model that provides a biochemical environment unique to the cornea, manufactured through a tissue printing process using dERS, according to an exemplary embodiment of the present invention, wherein

FIG. 13 shows a curved cornea 3D printed based on an anatomical image of an eyeball (scale bar: 5 mm) according to an exemplary embodiment of the present invention, and

FIG. 14 shows the change in the state of dissolution of the Ru/SPS photoinitiator in the cornea printed according to FIG. 13 over time.

FIG. 15 shows the structure of a cardiac model that provides a 3D-printed heart with a specific biochemical environment of the heart based on an anatomical image of the heart (scale bar: 5 mm) according to an exemplary embodiment of the present invention.

FIG. 16 demonstrates the live/dead cell fluorescence image results over the time (5 seconds, 3 minutes, and 10 minutes) exposed to visible light for hdERS prepared by mixing 2% hdECM and 1/10 mM Ru/SPS according to an exemplary embodiment of the present invention.

FIG. 17 is a graph showing the number of dead cells per unit area, calculated from the image results in FIG. 16.

FIG. 18 shows the change in the composite modulus value over time for hdERS made by mixing 2% pure hdECM with 0.5/5 mM, 1/10 mM, and 2/20 mM Ru/SPS, respectively, according to an exemplary embodiment of the present invention.

FIG. 19 is a photograph showing gel formation through photopolymerisation for the natural biomaterial (hdECM) according to an exemplary embodiment of the present invention, gelatin (another natural biomaterial and PEGDA (a synthetic compound material).

FIG. 20 is a graph comparing the complex modulus before and after light irradiation for the above-mentioned natural biomaterials (hdECM), gelatin, and synthetic compound material PEGDA.

FIG. 21 shows the results of the live/Dead assay performed on the above-mentioned natural biomaterials (hdECM), gelatin (Gelatin), and synthetic compound material PEGDA to evaluate biocompatibility.

FIG. 22 shows the CCK-8 results of absorbance for the above-mentioned natural biomaterials (hdECM), gelatin (Gelatin), and synthetic compound material PEGDA.

### DETAILED DESCRIPTION

The invention is subject to various modifications and can have many different embodiments, some of which are illustrated in the drawings and described in the accompanying detailed description. However, this is not intended to limit the invention to any particular embodiment, and should be understood to include all modifications, equivalents or substitutions that fall within the scope of the present idea and technology. In describing the invention, where it is believed that a detailed description of the relevant prior art would obscure the gist of the invention, such detailed description is omitted.

The terminology used in this application is intended to describe particular embodiments only and is not intended to limit the invention. Singular expressions include the plural unless the context clearly indicates otherwise. In this application, the terms such as ‘comprising’ or ‘including’ are used to indicate the presence of the features, numbers, steps, operations, components, parts, or combinations thereof described in the specification and are not intended to preclude the possibility of the presence or addition of one or more other features, numbers, steps, operations, components, parts, or combinations thereof.

Terms such as first, second, and the like may be used to describe various components, but the components are not to be limited by such terms. They are used only for the purpose of distinguishing one component from another.

The present invention, according to an exemplary embodiment, may comprise the method for preparing a bioink composition. This method includes the steps of: preparing an extracellular matrix (ECM) by treating tissue separated from a living body with a decellularization process; adding a photoinitiator to the prepared decellularized extracellular matrix (dECM) to form a decellularized extracellular matrix (dECM) containing a photoinitiator (dERS), where the photoinitiator is ruthenium/sodium persulfate (Ru/SPS); and irradiating the photocrosslinking extracellular matrix (dERS) with visible light to photocrosslink it.

The present invention relates to a bioink composition for visible light curing, a method for preparing the same, and a printing method thereof. Specifically, the present invention relates to a method for preparing a bioink composition for visible light comprising mixing a ruthenium/sodium persulfate (Ru/SPS) as a photoinitiator with a decellularized extracellular matrix (dECM) obtained by treating decellularized tissue isolated from a living body and photocrosslinking the mixture by visible light irradiation.

First, the step of preparing the extracellular matrix (ECM) is to prepare the extracellular matrix (ECM) by treating the tissue separated from the living body with a decellularization process. The process or method for separating tissue from the living body, treating it with decellularization, and preparing extracellular matrix are not particularly limited and include various known techniques in this field of technology.

The term ‘extracellular matrix (ECM)’ used in this specification refers to a natural support for cell growth derived from decellularized tissues in in mammals or multicellular organisms. The extracellular matrix can undergo further processing, such as dialysis or crosslinking.

The extracellular matrix can be in various forms in mammals and can contain approximately 90% collagen. Extracellular matrices derived from various biological tissues may differ in their overall structure and composition due to the unique role each tissue requires. ‘Derive’ and “derived” above refer to ingredients obtained from the source mentioned by a useful method. For example, an extracellular matrix-derived gel refers to a gel containing extracellular matrix components obtained from a tissue by various technically known methods for isolating the extracellular matrix. Alternatively, the tissue-derived extracellular matrix may refer to an extracellular matrix that contains components obtained from a specific tissue by a useful method.

The extracellular matrix may be a mixture of structural and non-structural biomolecules, including but not limited to collagen, elastins, laminins, glycosaminoglycans, proteoglycans, antimicrobials, chemoattractants, cytokines, and growth factors. The tissue may be selected from a group consisting of, but not limited to, brain, optic cup, kidney, liver, pancreas, neural tube, gastrointestinal tract, large intestine, prostate, breast, heart, salivary gland, endometrium, mammary gland, thyroid, tongue, and lung.

The above decellularized extracellular matrix (dECM) may be removed of more than 95% of the tissue cells. In one embodiment, the tissue may be agitated in a decellularization solution for 3 to 24 hours, and more than 95% of the tissue cells may be removed by the decellularization. The ‘decellularization solution’ may include various detergent components for removing tissue cells, such as, for example, hypertonic saline, peracetic acid, Triton-X, SDS, or other detergent components, but is not limited to these.

The above-mentioned decellularized extracellular matrix (dECM) can be dried, such as freeze-drying or air-dring. The dried extracellular matrix can be subdivided by a method that includes steps of tearing, milling, cutting, grinding, and shearing. The above-mentioned subdivided extracellular matrix can be processed into a powder form by methods such as grinding or milling, either in a frozen state or in a freeze-dried state. This decellularized extracellular matrix (dECM) is considered a biomaterial for mimicking and reproducing the complex microenvironment within tissue-mimicking constructs because it has a composition and formulation of biomolecules similar to those of actual tissues and organs. However, existing dECM-based bioinks remain insufficiently printable and mechanically stable, making them difficult to apply practically in the production of 3D tissue models. Therefore, methods such as adding external crosslinkers to dECM-based bioink or the introduction of a photocrosslinking process through chemical modification of dECM-based bioink have also been attempted. However, these methods are limited in that the existing bioactivity of dECM is inevitably reduced.

In this situation, the inventors confirmed that di-tyrosine bonding can be generated between tyrosine residues in the extracellular matrix (dECM) by using ruthenium/sodium persulfate (Ru/SPS) as a photoinitiator and photocrosslinking with visible light in decellularized extracellular matrix (dECM), and then completed the present invention.

Accordingly, the present invention comprises the following steps: preparing an extracellular matrix for photo crosslinking (dERS) by mixing ruthenium/sodium persulfate (Ru/SPS) as a photoinitiator with the above-mentioned decellularized extracellular matrix (dECM) and irradiating the above-mentioned extracellular matrix for photo crosslinking (dERS) with visible light to photo crosslink it.

In one embodiment, the ruthenium/sodium persulfate (Ru/SPS) may be a mixture of ruthenium and sodium persulfate pre-mixed, and it is also possible to use pure ruthenium and sodium persulfate, respectively. The mixing or usage ratio of the ruthenium and sodium persulfate is not particularly limited. For example, the ruthenium and sodium persulfate may be mixed in a ratio of 1:10 to 10:1.

The term ‘extracellular matrix for photo crosslinking (dERS) or dERS’ used in this specification refers to a mixture of ruthenium/sodium persulfate (Ru/SPS) as a photoinitiator in a decellularized extracellular matrix (dECM).

The visible light-induced crosslinking method using ruthenium/sodium persulfate (Ru/SPS) as a photoinitiator in decellularized extracellular matrix (dECM) according to the present invention has the following advantages. First, the photochemical crosslinking reaction within the visible light range (400-450 nm) can prevent DNA damage in cells compared to the crosslinking reaction using UV (350-400 nm). Secondly, the high molecular absorption coefficient of visible light causes an efficient crosslinking reaction even at relatively low initiator concentrations. Third, the deep penetration depth of visible light is advantageous for curing large-volume structures.

The ruthenium (Ru) photoinitiator has already been applied to natural biomaterials such as fibrin, collagen, and silk. According to the present invention, in the presence of ruthenium (Ru), visible light, and an electron acceptor such as sodium persulfate (SPS), Ru²⁺ first is photolyzed into Ru³⁺. The Ru³⁺ generated in this way can oxidize aromatic residues including tyrosine present in the extracellular matrix. The oxidized tyrosine group generates tyrosyl free radicals, which are consumed by forming di-tyrosine bonds between nearby tyrosine residues (see FIG. 2). Accordingly, one embodiment of the present invention may be that di-tyrosine bonding is generated between tyrosine residues in the extracellular matrix (dECM) by the above-mentioned visible light irradiation.

Considering that tyrosine-carrying proteins are abundantly contained in dECM bioink, the inventors hypothesized that visible light-induced crosslinking using Ru/SPS photoinitiators could quickly and cell-friendly crosslink dECM bioink. Such a visible light-based photocurable dECM as a bioink for 3D bioprinting has high shape retention and has considerable potential for producing dECM-based tissue mimics with large volume and complex structure, which can be confirmed in the following exemplary embodiments.

In the present invention, the concentration or amount of ruthenium/sodium persulfate (Ru/SPS) mixed into decellularized extracellular matrix (dECM) as the photoinitiator is not particularly limited. However, it is possible to mix ruthenium/sodium persulfate (Ru/SPS) as the photoinitiator with a concentration in the range of 0.5/5 mM to 10/100 mM in decellularized extracellular matrix (dECM), and it is desirable to mix it with a concentration in the range of 1/10 mM to 2/20 mM. If it is below the above range, it is difficult to manufacture a stable structure due to insufficient photo crosslinking effect, and if it exceeds the above range, cell compatibility may be reduced due to the oxidation ability of Ru³⁺.

In addition, the visible light may have a wavelength within the range of general visible light. For example, the visible light may have a wavelength within the range of 400 nm to 450 nm, and it is more desirable to have a wavelength within the range of 400 nm to 425 nm. If it is below the above range, the gelation of the bioink composition may not be well performed, and if it exceeds the above range, the gelation may not be well performed or cell compatibility may be poor.

In addition, the time for irradiating the above-mentioned visible light is not particularly limited, but it is possible to irradiate the above-mentioned visible light for a period ranging from a few seconds to tens of minutes. For example, the time to irradiate the visible light may be performed for a time ranging from 5 seconds to 10 minutes, preferably for a time ranging from 1 minute to 5 minutes, and more preferably for a time of 3 minutes. If the duration of the visible light irradiation is less than the above range, gelation may not occur well, and if it exceeds the above range, gelation may not occur well or cell compatibility may be poor.

In addition, the present invention may further include a step of thermally crosslinking the above-mentioned photocrosslinked extracellular matrix (dERS). Thermal crosslinking can be performed at a temperature above room temperature for 10 minutes to 3 hours. In this way, performing thermal crosslinking after photocrosslinking in sequence has the effect of further strengthening the crosslinking network, thereby increasing shape retention.

Another embodiment of the present invention relates to a printing method of a bioink composition, the method comprising the following steps of: preparing an extracellular matrix (ECM) by treating tissue separated from a living body with a decellularization process; mixing the prepared decellularized extracellular matrix (dECM) with ruthenium/sodium persulfate (Ru/SPS) as a photoinitiator to produce a decellularized extracellular matrix (dERS) for photo crosslinking; and printing the above-mentioned photocrosslinkable extracellular matrix (dERS) in an environment where visible light is irradiated.

In other words, the steps of preparing the above extracellular matrix (ECM) and preparing the above extracellular matrix for photo crosslinking (dERS) are the same as described above, and the printing method of the bioink composition includes the process of printing the above-prepared extracellular matrix for photo crosslinking (dERS) in an environment where visible light is irradiated.

The method of printing dERS can use the method of printing dECM. For example, it can be printed based on extrusion by a nozzle, and it is also possible to print it using the digital light processing (DLP) photopatterning method. However, it is possible to print dERS in an environment where visible light is irradiated, either before or after printing, or during the printing process.

Accordingly, it is possible to generate di-tyrosine bonding between tyrosine residues in the extracellular matrix (dECM) through the above-mentioned irradiation of visible light.

Still another embodiment of the present invention is a bioink composition comprising extracellular matrix (dECM) obtained by decellularization of tissue isolated from a living body, and ruthenium/sodium persulfate (Ru/SPS) as a photoinitiator, which can be photocrosslinked by irradiation with visible light.

In other words, the bioink composition includes extracellular r matrix (dECM) and ruthenium/sodium persulfate (Ru/SPS), and the bioink composition is characterized by being photocrosslinked by visible light irradiation. Then, the di-tyrosine bonding can be generated between tyrosine residues in the extracellular matrix (dECM) by the above-mentioned visible light irradiation. In addition, the ruthenium/sodium persulfate (Ru/SPS) may be included in the extracellular matrix (dECM) at a concentration ranging from 0.5/5 mM to 10/100 mM. This is described above.

Still another embodiment of the present invention is a method for producing a tissue-mimicking construct which includes the step of printing the bioink composition in an environment where visible light is irradiated.

The method or device for irradiating visible light is not particularly limited and may include various known in this technical field. The visible light may have a wavelength within the range of 400 nm to 450 nm.

As mentioned above, the bioink composition according to the present invention has excellent mechanical properties due to physical crosslinking through intermolecular interactions, and has excellent cell compatibility as well as high shape retention.

The technology of inducing the di-tyrosine crosslinking reaction by adding Ru/SPS photoinitiators to dECM according to the present invention enables the expansion of bioprinting tissue-mimicking construct by photocrosslinkable dERS. Engineered tissue construct of various sizes and shapes can be freely created, which can solve many of the problems encountered in tissue engineering and regenerative medicine. The photocrosslinking method of dERS can improve the printability of existing bioprinting systems and enable the enhancement of cell function and maturation of tissues by excluding the addition of light sources or substances that can cause cytotoxicity. In other words, the present invention not only provides structural stability so that the printed structure can maintain its previously designed shape, but also provides the ability to perform an excellent biofunctional role as a engineered tissue constructs. This has enormous potential for downstream clinical applications such as tissue regeneration for disease treatment and disease models for in vitro tissue and drug testing. In addition, the present invention confirms that the photocurable dERS can be applied to both extrusion-based printing systems and DLP systems, which is expected to have great potential in the field of biofabrication.

The present invention can be better understood by the following exemplary embodiments, which are for illustrative purposes only and are not intended to limit the scope of protection as defined by the attached patent claims.

### Exemplary Embodiments

### 1. Preparation of Bio-Ink Composition

1-1. hdECM (Heart Decellularized ExtraCellular Matrix) Powder Manufacturing

The porcine left ventricle was separated from the whole heart and decellularized according to a previously known protocol. In other words, the heart tissue was cut into 1 mm thick slices and stirred in tap water for one hour to remove blood. After that, it was treated with a 1% sodium dodecyl sulfate anionic surfactant solution for 60 hours and then with 1% Triton X-100 nonionic surfactant diluted in phosphate-buffered saline. The cells-removed tissue was soaked in isopropyl alcohol for 2 hours. The treated tissue was immediately washed with 1× phosphate-buffered saline for 72 hours to remove residual detergent. After that, the tissue was sterilized by soaking it in 0.1% peracetic acid diluted in 4% ethyl alcohol for 3 hours, and then washed with 1× phosphate-buffered saline. The powder obtained in this way was stored at −80° C. until it was used later.

### 1-2. Co-dECM (Cornea Decellularized ExtraCellular Matrix) Powder Manufacturing

The entire cornea was dissected from the bovine eyeball and washed with phosphate-buffered saline containing penicillin (100 U mL⁻¹) and streptomycin (0.1 mg mL⁻¹). The stromal layer was separated from the cornea and soaked in 20 mM ammonium hydroxide containing 0.5% Triton X-100 for four hours. Then, it was stored in a storage-stable Tris-HCl buffer for 24 hours and treated in a 10 mM Tris-HCl solution containing 1% Triton X-100 at 37° C. for 24 hours. The Co-dECM tissue was sterilized by treating it with 1% peracetic acid diluted in 50% ethanol for 10 hours, and after the de-cellularization process, it was freeze-dried overnight and then cryogenically decomposed into a fine powder using liquid nitrogen and a milling machine and stored at −80° C.

### 1-3. DECM Preparation of Bio-Ink

The powder samples of each Co-dECM and hdECM prepared in the above-mentioned embodiments 1-1 and 1-2 were placed in a 0.5 M acetic acid solution containing pepsin powder and dissolved at room temperature for 72 hours. The solution containing the dECM powder was filtered using a 40 μm pore mesh and stored at 4° C. Before the experiment, each Co-dECM and hdECM solution was neutralized (pH 7.4) using a 10 N NaOH aqueous solution. In addition, 10× phosphate buffered saline and sterile distilled water were added to the neutral hdECM solution.

### 1-4. DERS Bioink Manufacturing

Ru/SPS (0.5/5 mM, 1/10 mM, 2/20 mM) was mixed with each of the 2% dECMS prepared in embodiments 1-3 above. Photocrosslinking was carried out by exposing the gel to visible light (30 mW cm⁻²) with a wavelength in the range of 400-450 nm for 3 minutes, and then placing it at 37° C. for 1 hour to thermally crosslink it to produce a gel structure.

### 2. Evaluation of the Gelation Properties of Bio-Ink Compositions by Light-Initiated Gelation

Photocrosslinking was carried out by mixing the 2% dECM prepared in embodiments 1-3 with Ru/SPS ( 2/20 mM) and exposing it to visible light (30 mW cm⁻²) with a wavelength in the range of 400-450 nm for 3 minutes.

As a comparative example, photocrosslinking was carried out by exposing 2% dECM prepared in embodiments 1-3 to visible light (30 mW cm⁻²) with a wavelength in the range of 400-450 nm for 3 minutes, with 0.5 wt % of LAP (Lithium phenyl-2,4,6-trimethylbenzoylphosphinate), 0.5 wt % of Irgacure 2959, and 2 mM of riboflavin, respectively, as conventional photoinitiators. In addition, Irgacure 2959 is known to be suitable for crosslinking in the 350-400 nm range of UV rather than visible light, so photocrosslinking was also carried out in UV (350-400 nm).

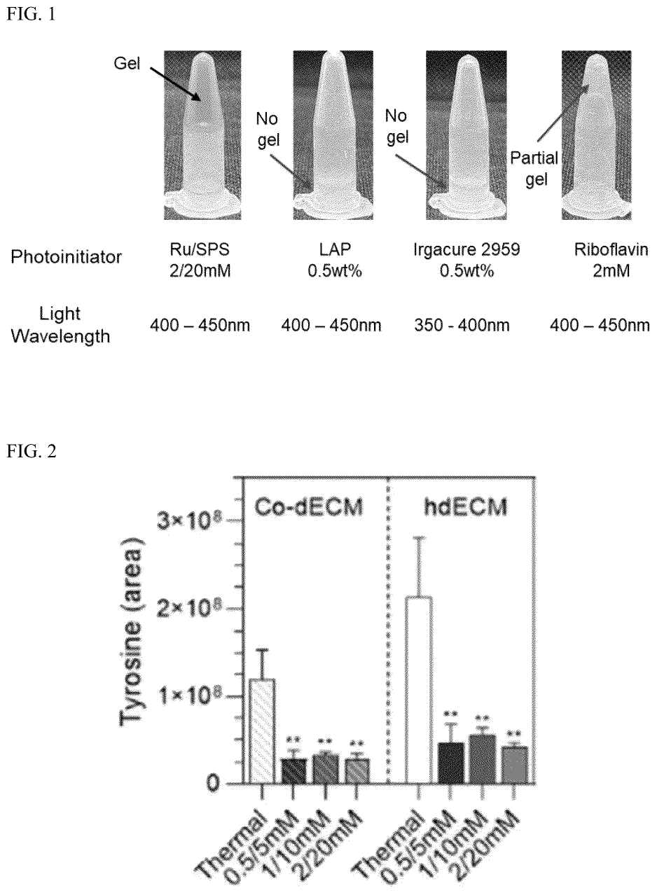

FIG. 1 shows the gelation properties resulting form photocrosslinking using Ru/SPS ( 2/20 mM) as a photoinitiator, with LAP, Irgacure 2959, and riboflavin used as a comparison, according to one embodiment of the present invention. As shown, gelation was successfully achieved when Ru/SPS was used as a photoinitiator, while no gelation occurred with LAP or Irgacure 2959. Partial gelation was observed when riboflavin was used. Additionally when riboflavin was crosslinked under visible light with a wavelength in the range of 400-450 nm, gelation did not occur either.

### 3. Evaluation of the Characteristics of Bio-Ink Compositions

3-1. Measurement of Autofluorescence from Di-Tyrosine in dECM

The gelated structure manufactured according to the above-mentioned embodiments 1-4 was placed under UV light with a wavelength of 320 nm and photographed with an iphone 11 Pro. It was converted to black and white using the GNU Image Manipulation Program (GIMP 2.10.22).

### 3-2. Sol Ratio and Swelling Ratio

The gelled structure manufactured according to the above embodiment 1 was prepared as a hydrogel sample. After photocrosslinking and/or thermal crosslinking, the weight of all samples was measured and recorded as M_{iw}. Three specimens were lyophilized directly under each condition. Another three samples obtained under each condition were immersed in phosphate-buffered saline at 37° C. overnight. The swollen samples were weighed as Mₛ, freeze-dried as M_{d}, and then weighed again.

The actual polymer weight fraction was calculated using Equation 1 below.

$$m \% = {M}_{id} / {M}_{iw} \qquad Equation 1$$

The sol fraction (M_{sol fraction}) and swelling ratio (q) were calculated using Equations 2 and 3 below.

$${M}_{sol fraction} = 100 \times ( {M}_{iw} \times m \% - {M}_{d} ) / ( {M}_{iw} \times m \% ) \qquad Equation 2$$

$$q = {M}_{s} / {M}_{d} \qquad Equation 3$$

### 3-3. Liquid Chromatography Tandem Mass Spectrometry (LC-MS/MS)

The gelated structure manufactured according to the above embodiment 1 was prepared as a sample and used for LC-MS/MS analysis. The prepared sample was hydrolyzed by treating it with 4M methanesulfonic acid containing 1 wt % phenol in a nitrogen environment for 18 hours. During the hydrolysis process, solid phase extraction (SPE) using a Strata C18-E (55 μm, 70 Å) cartridge was performed to remove the acid and separate it by dissolving it in 80% methyl alcohol. The eluate was then dried and reconstituted with 0.1% formic acid for analysis. The reverse-phase HPLC-MS/MS experiment was performed using a 2.6 μm Kinetex C18 100 Å column (150×2.1 mm) and an Agilent 1290 binary pump (200 μL min⁻¹). The sample was treated with 2% water-soluble acetonitrile containing 0.1% formic acid for 4 minutes, then increased to 80% acetonitrile over more than 3 minutes, and then equilibrated with the starting eluent for more than 3 minutes. The analytes were transferred to a QTRAP 6500 mass spectrometer and detected in multiple reaction monitoring mode using the positive ion mode. The ion spray was set to 5.5 kV and the temperature was set to 600° C. Nitrogen was used as the collision gas and the collision energy was set to 25%. The area of the peak was calculated using the analysis software v 1.6.2 (Sciex).

### 3-4. Rheological Test

After preparing the gelated structures manufactured according to the above embodiment 1 as samples, a stable shear rate change analysis was performed on each bio-ink sample at 4° C. to evaluate the viscosity. The complex modulus of the hydrogel samples after printing at 37° C. was measured using time-domain analysis. All hydrogel samples were exposed to visible light for three minutes before performing the time-dependent analysis. All measurements were performed three times.

### 3-5. Mechanical Property Inspection

A gelated structure manufactured according to the above embodiment 1 was prepared as a sample. Tensile tests were performed using a universal material testing machine with a maximum load and load resolution of 500×10⁻³ and 50×10⁻⁹ N, respectively. Structures made of pure dECM and dERS mixed with 0.5/5 mM, 1/10 mM, and 2/20 mM Ru/SPS were washed twice with phosphate-buffered saline for 5 minutes. The structures were then fixed between two clamps and paper was used as a geometric constraint. The paper was removed from the clamps and the sample was preloaded to eliminate slack and stretched at a fixed strain rate of 0.005⁻¹ s⁻¹ until failure. The elastic strain energy density and elastic modulus were calculated from the slope and area of the linear region of the stress-strain curve, respectively. In particular, the elastic strain energy density (Ur) was calculated using Equation 4.

$${U}_{r} = ( {σ}_{y} * {ε}_{y} ) / 2 , \qquad Equation 4$$

where σ_{y} is the yield stress and ε_{y} is the yield strain. In the compression test, the compression module was measured using the MTS Criteria 42 mechanical tester with a 5N load cell, and was measured in the linear region (10-15%) of the stress-strain curve. The hydrogel samples were prepared as in the self-luminescence measurement experiment and were additionally stored in phosphate-buffered saline at 37° C. overnight before testing. During the mechanical tests, the samples were placed parallel to the long axis and compressed at a constant crosshead speed of 0.01 mm s⁻¹. The experiment was performed at room temperature and the preload setting was 0.1 N.

### 3-6. Evaluation Results

As mentioned above, the inventors developed a new visible light-mediated crosslinking method by introducing Ru/SPS photoinitiators into dECM-based bioinks. The mechanism of this crosslinking reaction in dECM-based bioinks was investigated, and its versatility was demonstrated by controlling the ratio of the di-tyrosine synthesis produced after the crosslinking reaction. In particular, this crosslinking method was applied to corneal- and/or cardiac-derived dECM bioinks to produce 3D tissue models with a multi-scale hierarchical structure. These two types of dECM from different in vivo systems were selected in consideration of their contrasting properties related to blood vessels. For example, the cornea is a transparent tissue without blood vessels, while the heart is one of the vascular organs. The newly developed bioink (dERS) was prepared by mixing dECM and Ru/SPS photoinitiators.

dERS is crosslinked through a mechanism that is activated and oxidized by visible light (400-450 nm). When exposed to visible light, tyrosyl free radicals are generated, which form di-tyrosine bonds between nearby tyrosine residues and finally form a crosslinked network.

FIG. 2 shows the LC-MS/MS analysis results of tyrosine groups with or without photocrosslinking according to one embodiment of the present invention (**p<0.01). Tyrosine, one of the essential amino acids, exists as a useful residue that regulates the structural conformational transition of proteins. The most abundant protein in both types of dECM is collagen, which is known to contain tyrosine in most cases. LC-MS/MS analysis was used to identify and quantify tyrosine in dECM. Compared to the control (Thermal) without exposure to visible light, the invention shows that the amount of tyrosine in the dERS group is significantly reduced after exposure to visible light, indicating that a di-tyrosine bond is formed between the tyrosine residues (FIG. 2).

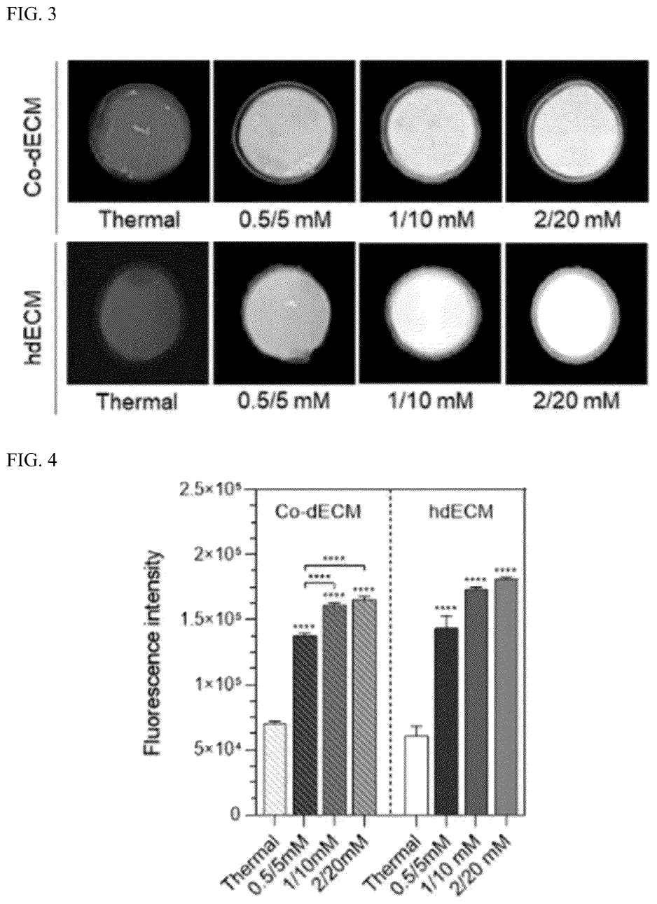

FIG. 3 is a self-fluorescence image of dECM by di-tyrosine as a result of the di-tyrosine crosslinking effect initiated by Ru/SPS according to one embodiment of the present invention, and FIG. 4 is the fluorescence intensity results of dECM and dERS bio-ink as a result of the di-tyrosine crosslinking effect initiated by Ru/SPS according to one embodiment of the present invention (***p<0.0001). To directly confirm the formation of the di-tyrosine bond in the dERS, the self-fluorescence intensity was measured when the sample was exposed to UV (320 nm). It has been reported that tyrosin with phenol groups emits autofluorescence in response to the absorption light at 320 nm. The captured fluorescence images indicate that the di-tyrosine binding in dERS has been formed (FIGS. 3 and 4). The dERS group with 2/20 mM of Ru/SPS showed the highest di-tyrosine content compared to the 0.5/5 mM and 1/10 mM groups. These results may be due to a more complex crosslinking mechanism, such as the formation of di-tyrosine in the dERS group with a relatively high concentration of Ru/SPS added. Therefore, it was thought that it would be interesting to conduct further experiments to explore the mechanism of the dERS crosslinking reaction. Nevertheless, we confirmed that di-tyrosine binding is one of the main factors that promote the crosslinking of dERS.

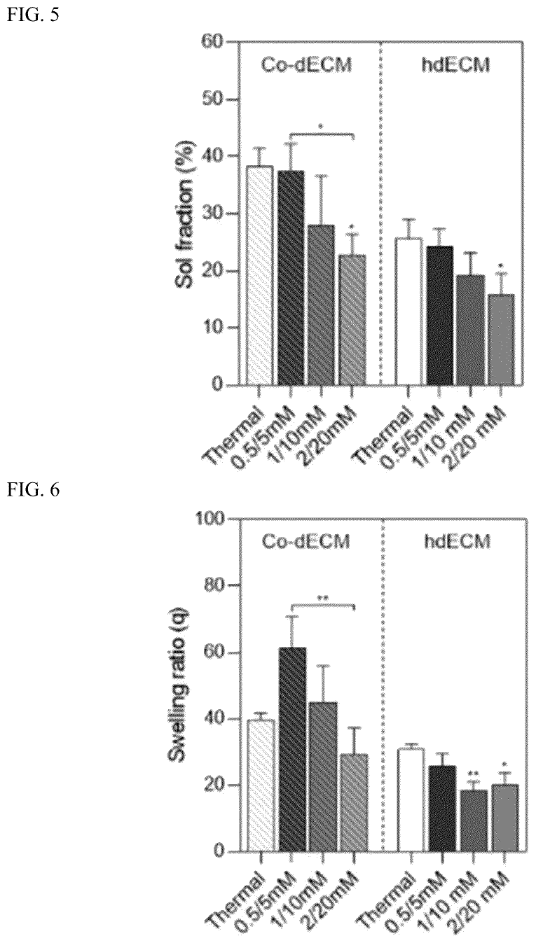

Next, the physicochemical properties of dERS were investigated through solubility and swelling ratio analysis. FIG. 5 shows the results of the sol fraction as the di-tyrosine crosslinking effect initiated by Ru/SPS according to one embodiment of the present invention (*p<0.1). The sol fraction, which is a measure of the polymer that did not participate in the crosslinking reaction, was found to be inversely proportional to the concentration of Ru/SPS, as shown in FIG. 5. These results are consistent with the results of the above fluorescence measurement data, which show that the more the concentration of Ru/SPS increases, the more the di-tyrosine bonds are formed.

FIG. 6 is the result of swelling ratio as a di-tyrosine crosslinking effect initiated by Ru/SPS according to one embodiment of the present invention (*p<0.1, **p<0.01). The swelling ratio was also found to be inversely proportional to the concentration of Ru/SPS (FIG. 6), which seems to be due to the tendency to decrease in the sol ratio, which is similar to the decrease in the sol ratio due to the formation of a denser crosslinked network as the crosslinking reaction becomes more active. We wanted to emphasize the development of dECM-based bioink, i.e. dERS, which uses the visible light-induced photo crosslinking method, which is very efficient and fast in that the crosslinking reaction is completed in seconds. First, dERS was capable of photocrosslinking regardless of pH conditions. Only the thermal crosslinking method was adopted in the existing dECM, which was only possible at a pH of around 7.4, where bonding could occur during the self-assembly process of the collagen component.

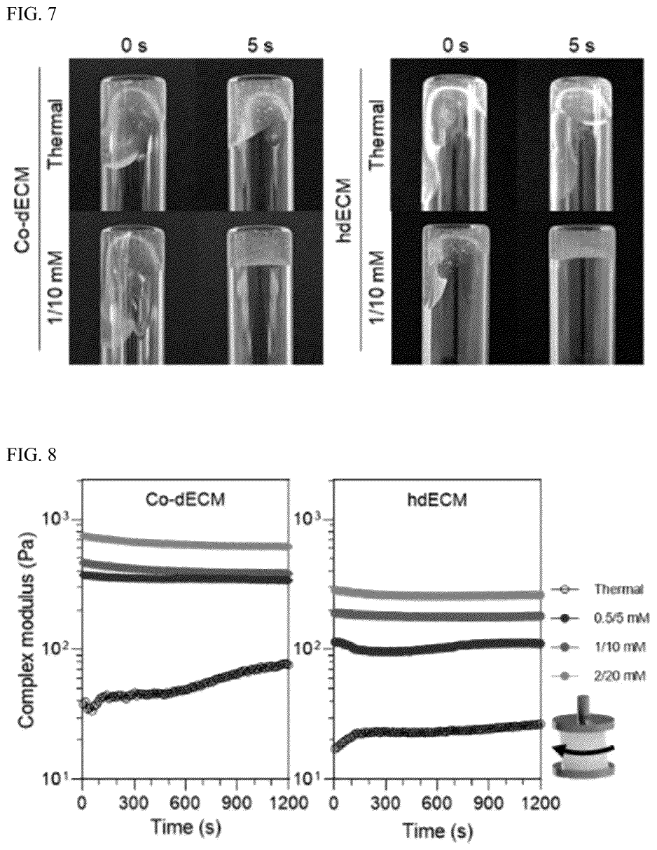

FIG. 7 shows the results of the crosslinking time in the photo-activated and thermally crosslinked dECM hydrogel as a result of the di-tyrosine crosslinking effect initiated by Ru/SPS according to one embodiment of the present invention. In this embodiment, it was confirmed that dERS could be converted into a gel state when exposed to visible light for 5 seconds at both pH 7.4 and 3.0 conditions, whereas dECM remained in the sol state (FIG. 7).

FIG. 8 shows the rheological properties of bioink before and after printing using 2% hdECM and 2% Co-dECM according to one embodiment of the present invention, which shows the results of increased storage and loss moduli after exposure to light for 3 minutes (light source: visible light (30 mW cm⁻² post-printing conditions)). The hdERS had up to 12.8 times higher composite modulus after three minutes of visible light exposure compared to hdECM (FIG. 8). It was confirmed that the new photocrosslinking method, which is applied in addition to the thermal crosslinking method applied to the existing dECM, not only improves the mechanical properties but also allows the value to be adjusted depending on the concentration of the photoinitiator.

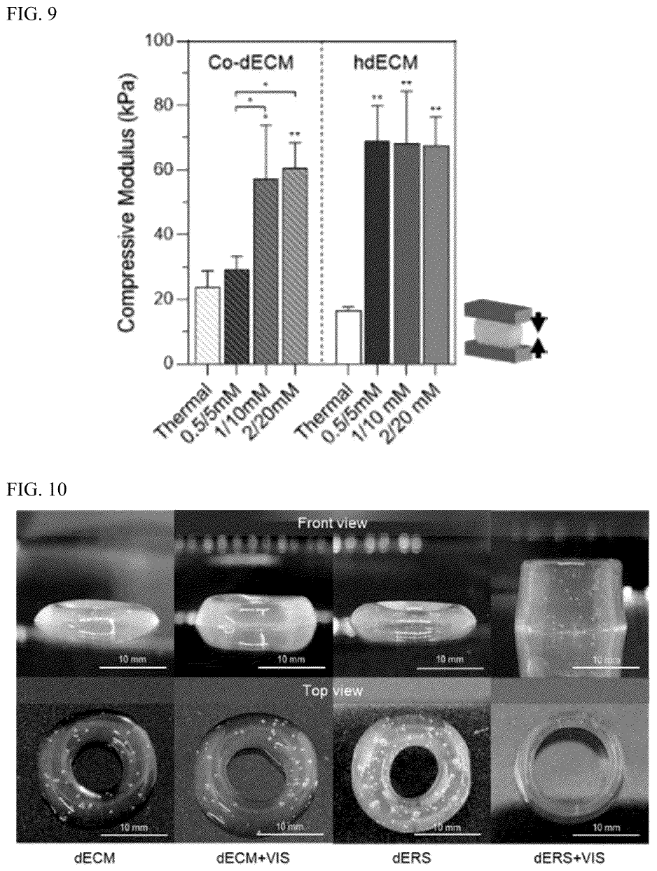

Furthermore, other types of mechanical properties of dERS were investigated using a universal testing machine (UTM). FIG. 9 shows the results of the rheological properties of bioink before and after printing using 2% hdECM and 2% Co-dECM according to one embodiment of the present invention, which shows the improvement in the mechanical properties of the compression modulus by the synthesis of di-tyrosine after photoactivation and thermo-crosslinking processes (*p<0.1, **p<0.01). Previously published research on dECM shows that dECM has a compression modulus in the range of approximately 0.18 to 3.0 kPa. In contrast, dERS has a compressive modulus of up to 86.4 kPa (for Co-dERS with 1/10 mM Ru/SPS added). In addition, for Co-dERS with 2/20 mM Ru/SPS added, the compressive modulus, elastic modulus, and recovery elastic modulus were 2.55, 3.79, and 20.04 times higher than those of Co-dECM, respectively (FIG. 9). Therefore, the formation of the di-tyrosine bond is considered to improve the rheological behavior and mechanical properties by increasing the tolerance limit of resistance to various types of stress. This improvement will ultimately contribute to better results for both printability and shape retention of dECM-based bioinks.

### 4. Fabrication and Evaluation of Structures by 3D Printing of Bioink Compositions

### 4.1. Extrusion-Based Printing

Visible light-activated dECM bioink was printed using a 3D bioprinting system in the laboratory. An LED module was installed at the bottom of the dispenser head to simultaneously perform extrusion and light curing. In accordance with embodiment 1 above, 2% hdECM or 2% Co-dECM bioink containing 2/20 mM Ru/SPS and neutralized with 10 M sodium hydroxide solution was placed in a syringe pre-cooled to 4° C. Different sized nozzles of 22G and 25G were used for the extrusion process. The range of applied air pressure was 20 to 50 kPa. The two extruded dERS were printed on a 40° C. printer stage and crosslinked under visible light irradiation from an LED module.

### 4.2. DLP Photopatterning

The DLP photopatterning experiment was performed using the CELINK Lumen X printer. The 1% hdECM solution according to embodiment 1 was neutralized with 10M sodium hydroxide solution. 1 mL of hdECM mixed with 1/10 mM Ru/SPS was placed in a Petri dish, and this Petri dish was subsequently placed on the printer stage. The 3D bioprinting process also included 5 million cells. The printing conditions were set to 50% light intensity, 30 s exposure time, and 100 μm penetration depth. After the printing process, the structure was washed with phosphate-buffered saline at 4° C. to remove uncrosslinked bio-reactive dERS.

### 4.3. Evaluation Results

As mentioned above, geometrically large and complex structures were produced using extrusion-based 3D bioprinting technology in dERS. FIGS. 10 to 12 show the results of 3D printing with a high aspect ratio structure at the centimeter level using a light-activated crosslinking system and dERS according to one embodiment of the present invention.

FIG. 10 is a schematic diagram illustrating the state in which di-tyrosine is synthesized by visible light activation by extrusion-based printing according to one embodiment of the present invention. An LED module was mounted at the bottom of the extrusion head for visible light irradiation, and this module was adjusted to irradiate visible light with an intensity of 30 mW cm⁻². The entire printing process was performed with visible light irradiation (wavelength 400-450 nm) (FIG. 10). To evaluate the shape retention of dERS, a cylindrical tube structure with a height of 5.5 mm and a diameter of 8.0 mm was designed, and the structure was printed using hdERS and compared with the previously created design.

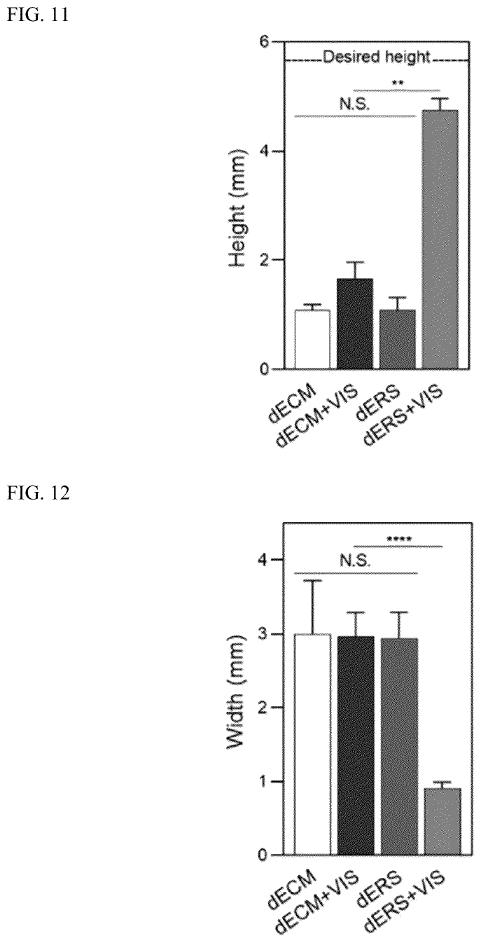

FIG. 10 is a comparative photograph of products manufactured using dECM dERS with or without a light-activated crosslinking system according to one embodiment of the present invention, and FIG. 11 is a result showing the height of the product in FIG. 10 (**p<0.01), and FIG. 12 is a result showing the width of the cylindrical structure in the product in FIG. 10 (****p<0.0001) (scale bar: 10 mm, VIS: visible light). As a result, only the dERS group (dERS+VIS) with visible light irradiation was able to reproduce up to 96.87% of the height in the design. In contrast, other groups (dECM, dECM+VIS, dERS) showed a relatively much lower height, exceeding a maximum of 22% of the height in the design (FIGS. 10 and 11). In addition, the dERS+VIS group had the narrowest line width, which is comparable to the inner diameter of the nozzle used, indicating that it produced a very precise and sophisticated structure. The other groups showed wider line widths than the dERS+VIS group, and as a result, it was confirmed that there was a very narrow empty area in the middle of the circle, indicating a significant difference in shape retention (FIG. 12).

### 5. Production and Evaluation of Tissue Analogs by 3D Printing of Bioink Compositions

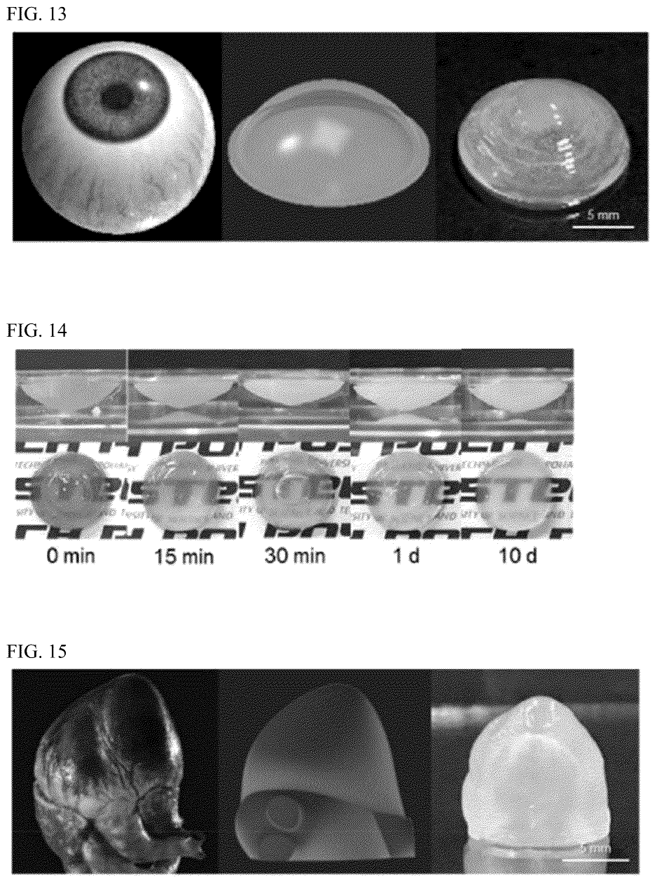

FIGS. 13 and 14 show an engineered corneal stroma construct that provides a biochemical environment unique to the cornea, produced by a tissue printing process using dERS according to one embodiment of the present invention, by the method according to embodiment 1 and 3.1.

FIG. 15 is a structure of an engineered heart tissue model that provides a biochemical environment unique to the heart, which is manufactured by a tissue printing process using dERS in accordance with one embodiment of the present invention, according to the above-mentioned embodiment 1 and 3.1.

### 5.1. Optical Transparency Characteristic Inspection

Corneal transparency was examined by light transmission measurement using a microplate reader. The printed corneal structure was prepared to a thickness of 200 μm. The light absorption value at a wavelength of 550 nm was measured using a microplate reader. Each sample was evaluated in three processes, and all transmittance results were corrected by measuring the absorbance of the absence of any sample.

### 5.2. Evaluation Results

As mentioned above, the purpose of this study was to demonstrate that tissue analogs printed with photocurable dERS according to the present invention have a degree of substrate remodelling and tissue regeneration similar to that of conventional dECM. Previously, Co-dECM has been used to provide cells that are encapsulated within the matrix or located in the vicinity with biological/biochemical signals unique to corneal tissue. However, Co-dECM does not have excellent shape retention, so it was difficult to maintain the curved surface structure of the designed corneal tissue even after thermo-crosslinking.

In this embodiment, a rapid photocrosslinking method was introduced to the Co-dERS made by mixing Ru/SPS and Co-dECM, which enabled the printing of a cornea-shaped structure including a curved surface (FIG. 13). FIG. 13 shows a curved cornea 3D-printed based on an anatomical image of an eyeball according to one embodiment of the present invention (scale bar: 5 mm), and FIG. 14 shows the change in the state of dissolution of the Ru/SPS photoinitiator in the cornea printed according to FIG. 13 over time. It was found that the transparency increased as the incubation time increased.

Similarly, we found that the photocurable hdERS, which is made by mixing hdECM and Ru/SPS, has sufficient shape retention to support cell growth and stably print heart-shaped structures. FIG. 15 is a heart-mimicking structure that provides a heart-specific biochemical environment and a 3D-printed heart based on an anatomical image of the heart (scale bar: 5 mm) according to one embodiment of the present invention. The 3D anatomical image of the heart for the production of the design was obtained from the NIH 3D Print Exchange, and a relatively simplified heart-shaped structure was printed based on this (FIG. 15). The heart-shaped structure was able to maintain its hollow structure stably.

### 6. Evaluation of Cell Compatibility of Bioink Composition by Light Irradiation Time

A gelated structure manufactured according to the above embodiment 1 was prepared and fabricated with dERS mixed with 1/10 mM Ru/SPS. Live/dead staining was performed on each bioink sample to evaluate the cell compatibility of the bioink over time under visible light irradiation.

In other words, the bioink was irradiated with visible light for 5 seconds, 3 minutes, and 10 minutes, depending on the group. After irradiation with visible light, the structures were washed twice with phosphate-buffered saline. Then, the live/dead cell staining reagent prepared according to the manufacturer's instructions was applied to the samples. After keeping it for 30 minutes, live/dead cell fluorescence images were obtained using a Nikon Eclipse Ti inverted microscope. Live cells are shown in green and dead cells are shown in red. Image J software was also used to calculate the number of dead cells per unit area.

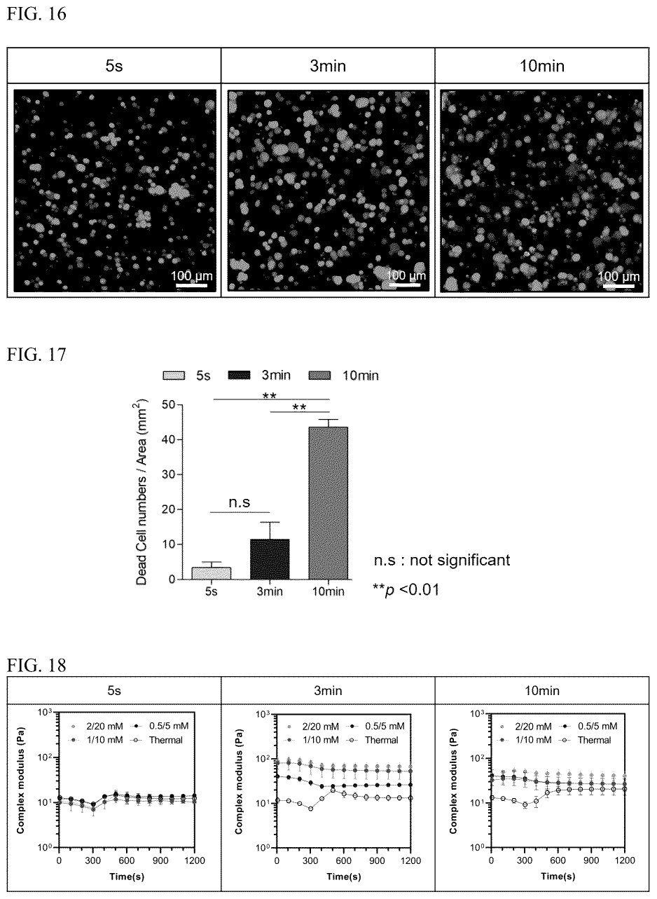

The results are shown as in FIGS. 16 and 17.

FIG. 16 is the result of live/dead cell fluorescence images according to the time (5 seconds, 3 minutes, and 10 minutes) of exposure to visible light for hdERS prepared by mixing 2% hdECM and 1/10 mM Ru/SPS according to one embodiment of the present invention, and FIG. 17 is a graph of the number of dead cells per unit area calculated from the image results of FIG. 16.

As shown in FIG. 16, the group exposed to visible light for 10 minutes had dead cells more easily observed than the groups exposed to visible light for 5 seconds and 3 minutes. And, as shown in FIG. 17, it was confirmed tha the number of dead cells increased significantly as the exposure time to visible light increased to 10 minutes, indicating that cell compatibility was reduced.

### 7. Evaluation of the Mechanical Properties of Bio-Ink Compositions by Light Irradiation Over Time

A crosslinked structure manufactured according to the above embodiment 1 was prepared as a sample, and the complex modulus of the sample was measured by performing a time-domain analysis in the same manner as in the above embodiments 3-4. In this case, the sample used was pure hdECM and hdERS mixed with 0.5 mM, 1/10 mM, and 2/20 mM Ru/SPS. Before performing the time-dependent analysis, the samples were exposed to visible light for 5 seconds, 3 minutes, and 10 minutes, respectively, depending on the group. Time-dependent analysis was performed using an Advanced Rheometric Expansion system (TA instrument, USA). All measurements were performed three times.

FIG. 18 shows the change in the complex modulus value over time when exposed to visible light for hdERS, which is a mixture of 2% pure hdECM and 0.5/5 mM, 1/10 mM, and 2/20 mM Ru/SPS, respectively, according to one embodiment of the present invention.

As shown here, hdERS showed no significant difference in the change in complex modulus value over time compared to hdECM after being exposed to visible light for 5 seconds.

On the other hand, when hdERS was exposed to visible light for 3 and 10 minutes, it had a significantly higher complex modulus than hdECM.

In addition, it was found that there was almost no change in the complex modulus value over time for both groups.

Furthermore, it was found that hdERS mixed with 1/10 mM Ru/SPS had a composite modulus value as good as that of hdERS mixed with 2/20 mM Ru/SPS.

### 8. Evaluation of Light Crosslinking Induction According to Biomaterials

Three types of Ru/SPS-based photoactive materials were prepared and divided into groups that were irradiated with light and those that were not, and the photo crosslinking induction performance was evaluated.

The same Ru/SPS initiator was used to photocrosslink natural biomaterials (hdECM) prepared according to embodiment 1, another natural biomaterial (gelatin), and a synthetic compound material, (poly(ethylene glycol) diacrylate (PEGDA)). After photocrosslinking, frequency sweep analysis was performed using an advanced rheometric expansion system (TA instrument, USA).

Specifically, the study focused on photocrosslinkable hdECM hydrogels (hdERS), prepared by mixing photoinitiator Ru/SPS with heart-derived decellularized extracellular matrix (hdECM) according to the present invention; photocrosslinkable gelatin hydrogels, prepared by mixing photoinitiator Ru/SPS with gelati;, and photocrosslinkable PEGDA hydrogels, prepared by mixing photoinitiator Ru/SPS with PEGDA. The concentration of the photoinitiator used was 0.5/5 mM, and the crosslinking was carried out in the same manner as described in embodiment 1.

Three types of samples (PEGDA+Ru/SPS, Gelatin+Ru/SPS, hdECM+Ru/SPS) were divided into those induced and those not induced by photo-crosslinking, and the complex modulus of each sample was measured using a frequency sweep analysis using an Advanced Rheometric Expansion system (TA instrument, USA). The angular frequency was increased from 0.01 to 100 rad/s⁻¹ under a constant shear strain of 0.1%. All experiments were conducted in triplicate.

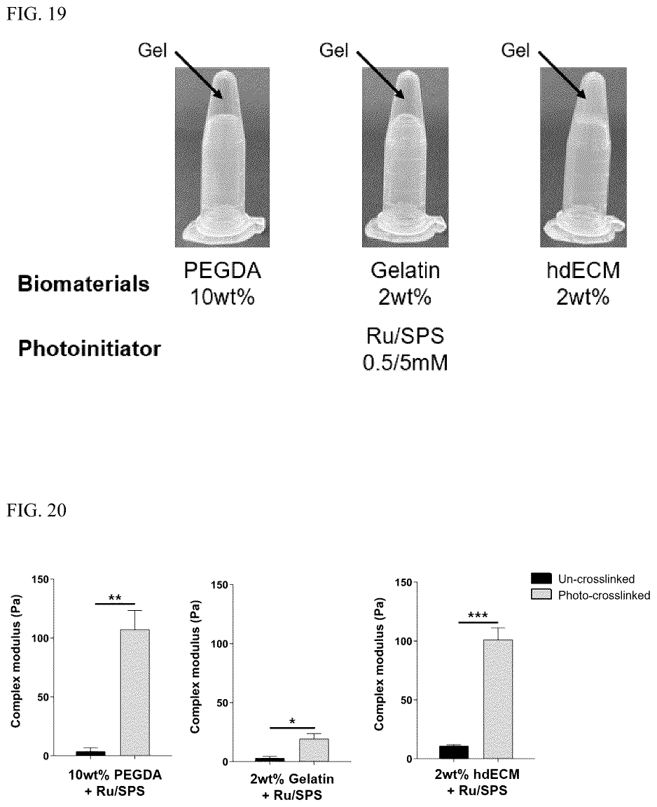

The results are shown in FIGS. 19 and 20. After visible light was irradiated, all materials were found to have been gelled by Ru/SPS-induced photocrosslinking (FIG. 19). In addition, comparing the complex modulus before and after irradiation, it was confirmed that all materials based on Ru/SPS according to the present invention had a high complex modulus after irradiation (FIG. 20).

### 9. Biocompatibility Evaluation According to Biomaterials

The biocompatibility of the three types of Ru/SPS-based photocurable materials prepared in embodiment 8 was evaluated using a live/dead assay using bone marrow-derived mesenchymal stem cells (BM-MSC) (cell concentration of 10⁷ cells/mL).

The experimental groups were as follows.

$$PEGDA + Ru / SPS + cells + light \qquad 1.$$

$$Gelatin + Ru / SPS + cells + light \qquad 2.$$

$$hdECM + Ru / SPS + cells + light \qquad 3.$$

After encapsulating cells in the three types of Ru/SPS-based photocurable biomaterials and culturing them for seven days, the results on day 1 and day 7 were compared. Specifically, cells were encapsulated in the three types of Ru/SPS-based photocurable biomaterials, crosslinked, and cultured for seven days, and the samples of each group were stained using the Live/Dead Viability Cytotoxicity Kit on days 1 and 7, following the manufacturer's instructions. Live/dead cell fluorescence images of the stained samples were obtained using a Nikon Eclipse Ti inverted microscope.

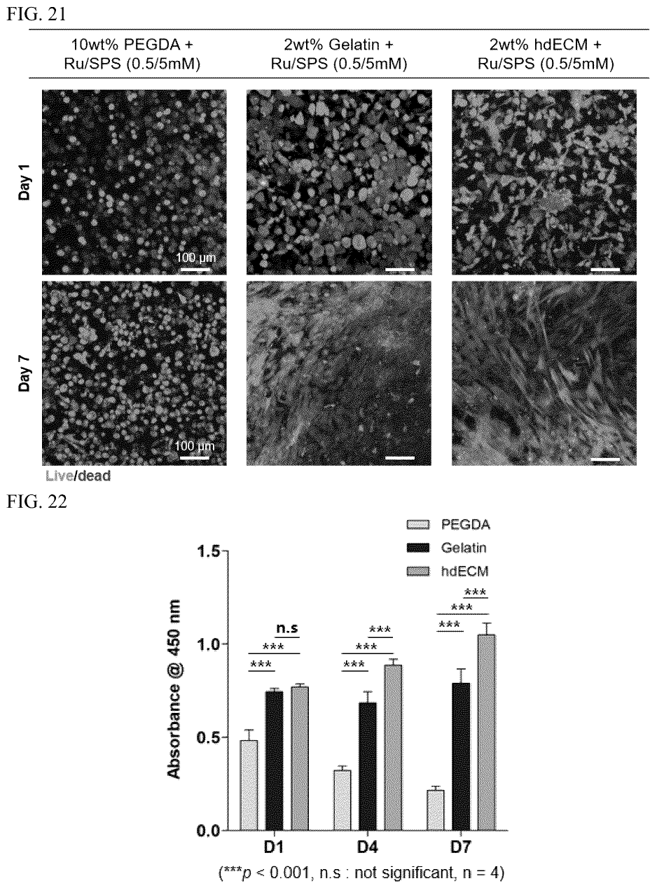

The results are shown in FIGS. 21 and 22. Live/dead staining results indicated that cells maintained high viability in all materials on day 1. In the imaging results from day 7, many cells with developed cell-specific morphology were observed in natural biomaterials such as gelatin and hdECM. However, cells with distinct shapes were not easily observed inside PEGDA (FIG. 21). According to the Cell Counting Kit-8 (CCK-8) results, hdECM was found to provide a significantly better cell-friendly environment for cell proliferation than gelatin. However, the absorbance value decreased in PEGDA, indicating that it does not provide an environment conducive to cell proliferation.

Although the above describes and explains the invention in relation to a specific preferred embodiment, it is evident to those skilled in the art that the invention can be modified and changed in various ways within the technical features or fields provided by the following patent claims.

1. A method for manufacturing a bioink composition, the method comprising the steps of:
preparing an extracellular matrix (ECM) by treating tissue separated from a living body with a decellularization process;
manufacturing decellularized Extracellular matrix Ruthenium Sodium persulfate (dERS) by mixing Ruthenium/Sodium PerSulfate (Ru/SPS) as a photoinitiator with the prepared decellularized extracellular matrix (dECM); and
irradiating visible light onto the manufactured decellularized Extracellular matrix Ruthenium Sodium persulfate (dERS) to photocrosslink the same.

2. The method of claim 1, wherein di-tyrosine bonding is generated between tyrosine residues in the extracellular matrix (dECM) by the above-mentioned visible light irradiation.

3. The method of claim 1, wherein ruthenium/sodium persulfate (Ru/SPS) is mixed as the photoinitiator with the decellularised extracellular matrix (dECM) at a concentration ranging from 0.5/5 mM to 10/100 mM.

4. The method of claim 1, wherein ruthenium/sodium persulfate (Ru/SPS) is mixed as the photoinitiator with the decellularised extracellular matrix (dECM) at a concentration ranging from 1/10 mM to 2/20 mM.

5. The method of claim 1, wherein the visible light has a wavelength in the range of 400 nm to 450 nm.

6. The method of claim 1, wherein the visible light has a wavelength in the range of 400 nm to 425 nm.

7. The method of claim 1, wherein the irradiation of the visible light is performed for a period ranging from 5 seconds to 10 minutes.

8. The method of claim 1, wherein the irradiation of the visible light is performed for a period ranging from 1 minute to 5 minutes.

9. The method of claim 1, wherein the irradiation of the visible light is performed for a period of 3 minutes.

10. The method of claim 1, wherein the step of thermally crosslinking the above-mentioned photocrosslinked extracellular matrix (dERS) is further included.

11. A printing method of a bioink composition, comprising the steps of:
preparing an extracellular matrix (ECM) by treating tissue separated from a living body with a decellularization process;
preparing an extracellular matrix for photocrosslinking (dERS) by mixing the prepared decellularized extracellular matrix (dECM) with ruthenium/sodium persulfate (Ru/SPS) as a photoinitiator; and
printing the above-mentioned photocrosslinkable extracellular matrix (dERS) in an environment where visible light is irradiated.

12. The method of claim 11, characterized in that di-tyrosine bonding between tyrosine residues in the decellularized extracellular matrix (dECM) is generated through the irradiation of visible light.

13. A bioink composition, comprising an extracellular matrix (dECM) obtained by treating decellularized tissue isolated from a living body with a photoinitiator and ruthenium/sodium persulfate (Ru/SPS), and capable of being photocrosslinked by visible light irradiation.

14. The bioink composition of claim 13, characterized in that di-tyrosine bonding is generated between tyrosine residues in the decellularized extracellular matrix (dECM) through the visible light irradiation.

15. The bioink composition of claim 13, characterized in that the ruthenium/sodium persulfate (Ru/SPS) is included in the decellularized extracellular matrix (dECM) at a concentration ranging from 1/10 mM to 10/100 mM.

16. A method for producing a tissue mimic comprising the bioink composition according to claim 13, wherein the bioink composition is printed in an environment where visible light is irradiated.

17. The method of claim 16, wherein the visible light has a wavelength in the range of 400 nm to 450 nm.
